(19) United States
(12) Patent Application Publication
(10) Pub. No.: US 2016/0281147 A1
(43) Pub. Date: Sep. 29, 2016

(54) ULTRASENSITIVE DIAGNOSTIC DEVICE USING ELECTROCATALYTIC FLUID DISPLACEMENT (EFD) FOR VISUAL READOUT

(71) Applicant: Xagenic Inc., Toronto (CA)

(72) Inventors: Justin D. Besant, Toronto (CA); Jagotamoy Das, Scarborough (CA); Ian B. Burgess, Toronto (CA); Wenhan Liu, London (CA); Edward Hartley Sargent, Toronto (CA); Shana O. Kelley, Toronto (CA)

(21) Appl. No.: 15/082,851

(22) Filed: Mar. 28, 2016

Related U.S. Application Data
(60) Provisional application No. 62/138,827, filed on Mar. 26, 2015.

(51) Int. Cl. C12Q 1/68; G01N 21/49; G01N 27/327; G01N 21/47

(52) U.S. Cl. CPC C12Q 1/6825; G01N 21/4788; C12Q 1/6834; G01N 21/49; G01N 27/3276

(57) ABSTRACT

Disclosed herein are methods and systems to detect low-concentration analytes by transducing small electrochemical currents into easily perceived, high-contrast visual changes using a new approach termed electrocatalytic fluid displacement (EFD)

### CROSS-REFERENCE TO RELATED APPLICATIONS

This claims priority to U.S. Provisional Application No. 62/138,827, filed Mar. 26, 2015, which is hereby incorporated herein by reference in its entirety. This application is related to PCT Application No. ______, filed Mar. 28, 2016 (Attorney Docket No. 109904-0026-WO1), which is hereby incorporated herein by reference in its entirety.

### BACKGROUND

Disposable, instrument-free testing devices are used routinely for home and physician office testing, but present-day devices lack sensitivity and are limited in applicability to a small class of highly abundant analytes. Direct, unambiguous visual readout is an ideal way to deliver a result on a disposable test device; however, existing readout approaches require the accumulation of a high level of an analyte, and therefore only abundant analytes have been detected visually, which can be difficult to interpret without sophisticated laboratory equipment. Developing ways to link a visible, unambiguous color change to rare biological molecules remains an unmet need. Recently, a variety of direct visual readout strategies have been reported: these include approaches based on nanoparticles, plasmonic nanomaterials, 2D materials, and enzymatic reactions. Unfortunately, these approaches require interpretation of subtle color changes. This can make analyses operator-dependent, or, in other cases, diminishes the benefits of a test being instrument-free benefits by requiring a scanner device. Strategies for direct colorimetric readout of electric currents include paper-based electrochromism, electrochromic polymers, metal oxides, and fluorescent dyes. Electrochromic polymers and dyes allow for rapid and reversible color switching in response to electrical currents, but the currents required to switch areas detectable to the naked eye are above the threshold necessary for sensitive electrochemical detection. Inducing visible color changes using currents below 1 microampere is a fundamental challenge; for such currents fail to supply enough electrons to electrochemically reduce a visibly-perceptible quantity of electrochromic material. Directly translating such low currents into visible changes has yet to be achieved without the aid of costly, power-consumptive active electronics such as amplifiers.

Developing new, easy-to-interpret interfaces that convey diagnostic results obtained with low-abundance analytes would enable the development of low-cost diagnostics for a spectrum of new diseases.

### SUMMARY

Disclosed herein are systems and methods for detecting biomolecular analytes and outputting the results of the detection to a point-of-care device. In one aspect, the system and methods disclosed herein provide an easy-to-interpret platform for visually presenting the detection results. The systems and methods are applicable to any biomolecular analyte, including analytes in very low concentrations. In one aspect, the new approach, which we term electrocatalytic fluid displacement (EFD), transduces a molecular binding event into an electrochemical current that drives the electrodeposition of a metal catalyst. The catalyst promotes the formation of bubbles (for example, within a chamber of an electrochemical assay) that displaces a fluid within a chamber of the device to reveal a high contrast change. The readout system may be coupled to a nanostructured microelectrode or any suitable electrode. In some implementations, the system may be used to directly, visually detect nucleic acid sequences at concentrations lower than about 1 pM in about 10 minutes (e.g., in less than about 20 minutes, in less than about 15 minutes, in about 10-minutes to about 12 minutes, or in 10 minutes or less). This represents the lowest limit of detection of nucleic acids reported to date using high contrast visual readout. The rate of detection for a given concentration of an analyte can be adjusted (e.g., slowed or accelerated) by adjusting the rate of formation of the bubbles. In some implementations, the growth of the bubbles can be adjusted, for example, by tuning the concentration of peroxide in the chamber. Although the systems and methods disclosed herein are exemplified using the detection of nucleic acids, they may be adapted for the detection of other biomolecular analytes, such as proteins and small molecules. See, e.g., Jagotamoy et al., Nature Chemistry, 4, 642-648(2012), which is incorporated by reference in its entirety.

According to one aspect, there is provided a detection system for detecting a target analyte in a sample, the system comprises a first chamber comprising a sensor electrode capable of presenting a biomolecular probe at the surface of the sensor electrode. The probe is capable of binding the target analyte. The system further includes a second chamber comprising a readout electrode electrically coupled to the sensor electrode, a peroxide, and a metal catalyst.

According to one aspect, which may be combined with any of the systems or methods described herein, there is provided a method for detection of a target analyte in a sample, the method comprising: providing a detection system comprising: a first chamber comprising a sensor electrode having a probe affixed thereto, said probe capable of binding the analyte; a second chamber comprising a readout electrode electrically coupled to the sensor electrode; contacting the sensor electrode with the sample; adding peroxide and a metal catalyst to the second chamber, either simultaneously or sequentially; monitoring a color change in the second chamber; wherein the color change in the second chamber is indicative of the presence of the target analyte in the sample.

According to a further aspect, there is provided a point-of-care diagnostic device configured to perform any of the methods described herein. The point-of-care device may include one or more of the systems described herein, either alone or in combination.

According to a further aspect, there is provided a kit comprising: a sensor electrode capable of presenting a biomolecule probe at the surface thereof, said probe capable of binding a target analyte; a readout electrode electrically coupled to the sensor electrode; a peroxide; and a metal catalyst. The kit may include one or more of the systems described herein, and may be used to perform any of the methods described herein.

In some implementations of the systems and methods provided herein, the sensor electrode is a nanostructured microelectrode. Other sensor electrode structures can also be used, including planar surfaces, wires, tubes, cones and particles. Commercially available macro- and micro-electrodes are also suitable. In some implementations, the readout electrode is a mesh or high-edge-density electrode. In some implementations, the sensor electrode is electrically coupled to the readout electrode through a platinum wire electrode.

In some implementations of the systems and methods provided herein, the peroxide and metal catalyst are added to the second chamber sequentially. In some implementations, the peroxide and metal catalyst are added to the second chamber simultaneously. In some implementations, the metal catalyst is platinum.

In some implementations of the systems and methods provided herein, binding of the target analyte to the probe on the sensor electrode generates an electrical current that results in electrodeposition of the metal catalyst on the readout electrode. In some implementations, electrodeposition of the metal catalyst on the readout electrode causes decomposition of the peroxide present in the second chamber which generates oxygen bubbles. In some implementations, the analyte is nucleic acid. In some implementations, the probe is nucleic acids or peptide nucleic acids (PNAs). In some implementations, generation of bubbles displaces a dye present in the peroxide solution. In some implementations, generated bubbles displace a dye present in the peroxide solution. In some implementations, the second chamber comprises a colored spot beneath the readout electrode. In some implementations, visual detection of the colored spot beneath the readout electrode indicates a color change. In some implementations, the second chamber comprises a lid comprising a diffraction grating, wherein generation of bubbles causes an index mismatch at the diffraction grating, causing a structural color change. In some implementations, the second chamber comprises a lid comprising a diffraction grating, wherein generated bubbles cause an index mismatch at the diffraction grating, causing a structural color change. In some some implementations, the second chamber comprises a lid comprising a photonic structure, wherein generation of bubbles induces the appearance or disappearance or reduction of incoherent scattering, coherent scattering or iridescence, causing a structural color change. In some implementations, the second chamber comprises a lid comprising a photonic structure, wherein generated bubbles induce the appearance or disappearance or reduction of incoherent scattering, coherent scattering or iridescence, causing a structural color change. In some implementations, the lid of the second chamber is made of material having an index of refraction substantially the same as the peroxide. In some implementations, detection of light diffraction into its component indicates a color change. In some implementations, detecting a change in iridescence indicates a color change.

In some implementations of the systems and methods provided herein, the first chamber comprises a redox reporter comprising Ru(NH₃)₆³⁺ and a reducing agent, wherein the reducing agent is not oxidizable or reducible by Ru(NH₃)₆³⁺ or Ru(NH₃)₆⁴⁺. In some implementations, the reducing agent is selected from: 3-mercaptopropionoic (MPA) acid, cysteamine (Cys), mercaptoethanol (MCE), cysteine, tris(2-carboxyethyl)phosphine (TCEP), and ethanolamine. In some implementations, the reducing agent comprises a combination of agents selected from: 3-mercaptopropionoic (MPA) acid+cysteamine (Cys); mercaptoethanol+cysteamine; cysteine+tris(2-carboxyethyl)phosphine (TCEP); ethanolamine+TCEP; cysteine+cysteamine; and ethanolamine+cysteamine.

In one aspect, this application provides a redox reporter system, comprising: a sensor electrode having a biomolecule probe affixed thereto, said probe is capable of binding a target analyte, such as a nucleic acid sequence; and an electrochemical redox reporter comprising Ru(NH₃)₆³⁺ and a reducing agent, wherein the reducing agent is not oxidizable or reducible by Ru(NH₃)₆³⁺ or Ru(NH₃)₆⁴⁺. In some implementations, the reducing agent is selected from: 3-mercaptopropionoic (MPA) acid, cysteamine (Cys), mercaptoethanol (MCE), cysteine, tris(2-carboxyethyl)phosphine (TCEP), and ethanolamine. In some implementations, the reducing agent comprises a combination of agents selected from: 3-mercaptopropionoic (MPA) acid+cysteamine (Cys); mercaptoethanol+cysteamine; cysteine+tris(2-carboxyethyl)phosphine (TCEP); ethanolamine+TCEP; cysteine+cysteamine; and ethanolamine+cysteamine. In such implementations, the redox reporter system further comprises a readout or detection unit.

In yet another aspect, there is provided a method of detecting a target analyte, such as a nucleic acid sequence. The method includes providing a sensor electrode having a biomolecule probe affixed thereto. The probe is capable of binding a target analyte, such as a nucleic acid sequence. The method further includes contacting a sample comprising the analyte to the sensor electrode, and contacting the sensor electrode with an electrochemical redox reporter. The redox reporter may comprise Ru(NH₃)₆³⁺ and a reducing agent. In some implementations, the reducing agent is not oxidizable or reducible by Ru(NH₃)₆³⁺ or Ru(NH₃)₆⁴⁺. The method further includes measuring a response signal from the sensor electrode using a readout or detection unit.

### BRIEF DESCRIPTION OF DRAWINGS

The patent or application file contains at least one drawing executed in color. Copies of this patent or patent application publication with color drawings will be provided by the Office upon request and payment of the necessary fee.

The foregoing and other objects and advantages will be apparent upon consideration of the following detailed description, taken in conjunction with the accompanying drawings, in which like reference characters refer to like parts throughout, and in which:

FIG. 1A shows the conversion of an electrochemical current from a nanostructured microelectrode into a visible change through the deposition of a catalyst that catalyzes bubble formation according to some implementations. In the example of FIG. 1A, as the bubble grew, the white dye was displaced to reveal a color, such as a blue color. Other visual indicia (including other color changes) may be used.

FIG. 1B provides an overview of colorimetric detection of ssDNA using electrocatalytic fluid displacement (EFD) according to some implementations. The target analyte hybridizes to a biomolecular probe, e.g., a complementary PNA probe. Ru(NH₃)₆³⁺ is electrostatically attracted to the negatively charged backbone of the target analyte, e.g., a nucleic acid sequence. A potential is applied to the NME which oxidizes Ru(NH₃)₆³⁺. The current resulting from the oxidation reaction is amplified using an electrochemical-chemical-chemical (ECC) reporter system. Ru(NH₃)₆³⁺ is regenerated by a first reducing agent, e.g., 3-mercaptopropionoic (MPA), which is in turn regenerated by a second reducing agent, e.g., cysteamine. The electrochemical current drives deposition of platinum, a catalyst for hydrogen peroxide decomposition, on a mesh electrode immersed in a solution containing platinum ions, e.g., Pe⁺. After the introduction of peroxide, bubbles form as the deposited platinum catalyzes the decomposition of the peroxide. The growing bubbles are transduced into a color change, for example, either through an optical density change or a structural color change. In the optical density approach, the bubble displaces a white dye to reveal a colored (e.g., blue) spot. To induce a structural color change, the peroxide solution with bubbles forming causes an index mismatch at a diffraction grating patterned in the underside of the chamber lid. Incident white light is diffracted into its component colors.

FIG. 1C provides an exemplary calculation of the time to visual appearance using electrocatalytic fluid displacement and reduction of an electrochromic compound as a function of applied current according to some implementations. In this example, the readout window is about a 200 μm×200 μm×50 μm chamber and the current is applied for about 10 s. The onset of bubble formation occurs as the solution is saturated with oxygen. A bubble is deemed to be visible once it reaches the volume of the chamber. The electrochromic dye in this example has the absorbance of malachite green and a visible change corresponds to a ΔOD of about 1 (where ΔOD is the change in optical density of the solution).

FIG. 2A shows bubble evolution as a function of time for various electrode geometries (as shown in the Figure) according to some implementations. In this example, platinum was deposited using about a 1 nA current for about 10 s. Bubble growth increases with the ratio of edges to surface area.

FIG. 2B shows average bubble area after about 20 minutes as a function of applied current using the electrodes with the highest mesh density according to some implementations. In this example, bubbles were confined to about a 50 μm tall channel.

FIG. 2C shows bubble growth as a function of time for various deposition currents using electrodes with the highest mesh density according to some implementations. In this example, bubbles did not form when no current is applied.

FIG. 2D shows images of bubble growth as a function of dye concentration acquired using an optical microscope according to some implementations.

FIG. 2E shows the transmission spectrum of the readout window before and after bubble growth according to some implementations.

FIG. 2F shows images of colorimetric readout as a function of deposition current and time according to some implementations. In this example, about 1 nA currents were detectable in about 5 minutes. The scale bar represents about 1 mm.

FIG. 3A shows spot size as a function of time for various deposition currents using electrodes with the highest mesh density according to some implementations. In this example, bubbles did not form when no current is applied. Error bars represent standard error.

FIG. 3B shows images of colorimetric readout as a function of deposition current and time using a diffraction grating according to some implementations. In this example, the window turned from optically transparent (which appears as black due to a black background) to cyan as light at that wavelength was diffracted towards the camera. About 1 nA currents were detectable in about 1 minute. The scale bar represents 1 mm.

FIG. 3C provides a comparison of the charge required to induce a visible color of a certain area and optical density change for a variety of readout strategies according to some implementations. The dashed line represents the calculated exposed area of a bubble generated using electrocatalytic fluid displacement. The dotted line represents the area of a monoatomic layer of platinum directly reducible by the current. In this example, the bubble was confined to about a 50 μm tall chamber, the reaction proceeded for about 10 min, and the ΔOD was about 1.

FIG. 4A shows electrochemical current as a function of time for various analyte concentrations after applying about 250 mV with respect to a Ag/AgCl reference electrode for about 3 s according to some implementations. Comparative data of electrochemical current for a non-target is also provided and shows lower magnitude of electrochemical current for target analyte.

FIG. 4B shows average peak electrochemical current as a function of analyte concentration according to some implementations. Data for a non-target is also provided for comparison.

FIG. 4C shows spot size as a function of target DNA concentration after about 10 minutes using dye displacement according to some implementations. About 1 pM ssDNA is detectable by eye. The visible threshold is defined as an area of about 200 μm×200 μm. Data for a non-target is also provided for comparison.

FIG. 4D shows images of the EFD device showing growth of the bubble over time as a function of analyte (ssDNA) concentration using dye displacement according to some implementations. Data for a non-target is also provided for comparison.

FIG. 4E shows spot size as a function of analyte (ssDNA) concentration after about 10 minutes using a structural color change according to some implementations. Data for a non-target is also provided for comparison.

FIG. 4F shows images of the EFD device showing growth of the bubble over time as a function of analyte (ssDNA) concentration using a structural color change according to some implementations. The scale bar represents about 1 mm. Error bars represent standard error. Data for a non-target is also provided for comparison.

FIG. 5 shows the effect of hydrogen peroxide concentration on bubble growth after about 2 minutes in peroxide solution of different concentrations for various applied currents according to some implementations. By tuning the peroxide concentration it is possible to control the rate of bubble growth. For example, as shown in FIG. 5 when about 3% peroxide was used, no bubbles formed within the about 3 minutes after applying about a 1 nA deposition current. However, bubbles formed within the same timeframe when about 10% peroxide was used.

FIG. 6 shows a diagram for a setup used for electrochemical sensing according to some implementations. The NME acts as the sensor electrode and the Au mesh readout electrode acts as the counter electrode. In this example, a platinum wire serves as an electronic bridge between the two solutions. The deposition solution, in this example, is K₂PtCl₄ solution. However, any suitable combination of assay solution and electrodeposition solution may be used.

FIG. 7 shows the effect of electroless deposition according to some implementations. Electrodes were immersed in about 30% H₂O₂ before and after dipping in a platinum solution for about 25 minutes (no potential was applied). There was no bubble formation even after about 10 minutes in either case, indicating that no appreciable electroless deposition occurred under the above experimental conditions.

FIG. 8 shows visible color change caused by palladium in the presence of peroxide and hydroquinone according to some implementations. After inducing PdCl₂ electrodeposition onto an electrode through about a 1000 nA current for about 10 s, the electrode was dipped in a solution of about 10 mM hydroquinone in about 30% H₂O₂. After about 20 minutes, the solution turned brown and bubbles formed. FIG. 8 is merely illustrative. It is understood that other values may be used. For example, the applied current may be reduced or increased, the duration of electrodeposition may be increased or reduced, and the concentration of the solutions used may be increased or decreased as needed.

FIG. 9 depicts electron transfer pathway in an Electrochemical-Chemical-Chemical (ECC) redox system according to some implementations, comprising oxidation of Ru(NH₃)₆³⁺ to Ru(NH₃)₆⁴⁺ on the electrode surface and regeneration of Ru(NH₃)₆³⁺ by a first reducing agent R1; oxidation of the first reducing agent R1 to R1(ox) and regeneration of R1 by a second reducing agent R2; and oxidation of a second reducing agent R2 to R2(ox). The EEC redox amplification enables a DC Readout with High Signal/Noise ratio.

FIG. 10 shows a comparison of ECC reporter systems using different reducing agents according to some implementations. Panel (A) shows a comparison of ECC reporter systems using different reducing agents at bare NMEs. (i) a: Mercaptopropionoic acid (MPA), b: MPA+ruthenium hexamine (RuHex), c: MPA+RuHex+cysteamine, d: MPA+RuHex+cysteamine+(tris(2-carboxyethyl)phosphine) (TCEP). (ii) a: Mercaptoethanol (MCE), b: MCE+RuHex, c: MCE+RuHex+cysteamine, d: MCE+RuHex+cysteamine+TCEP. (iii) a: L-cysteine, b: L-cysteine+RuHex, c: L-cysteine+RuHex+TCEP (iv) a: Ethanolamine+RuHex, b: Ethanolamine+RuHex+cysteamine.

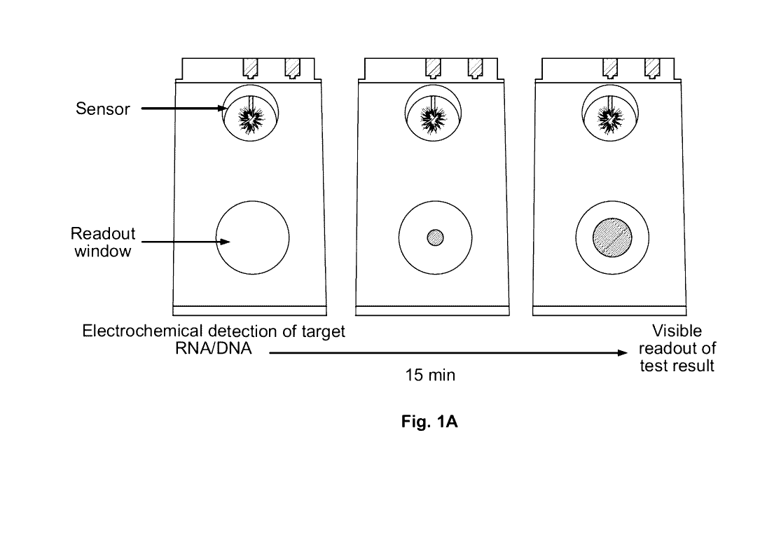

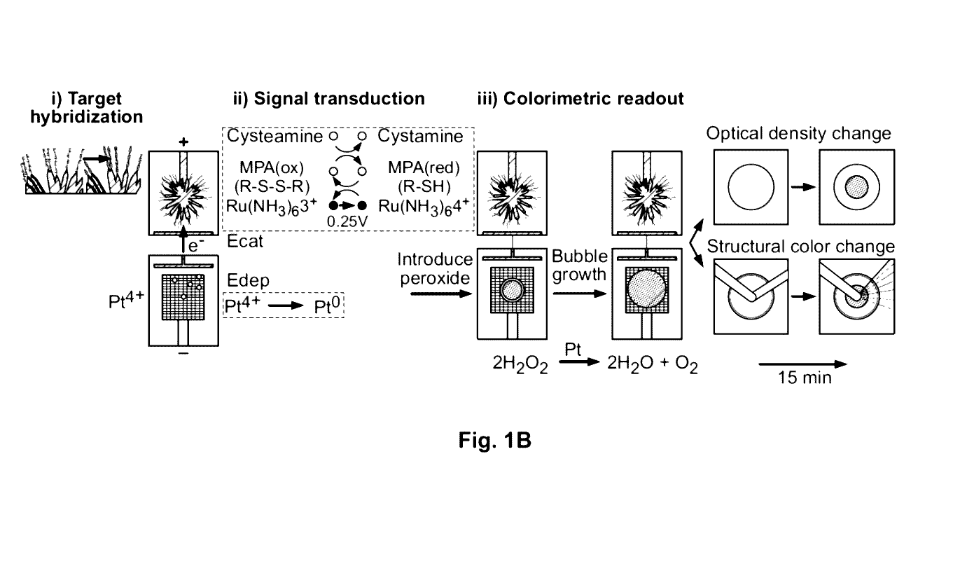

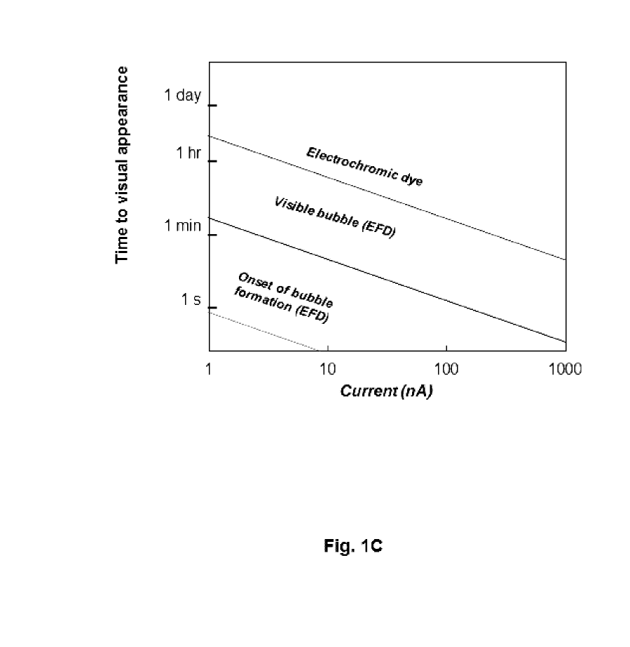

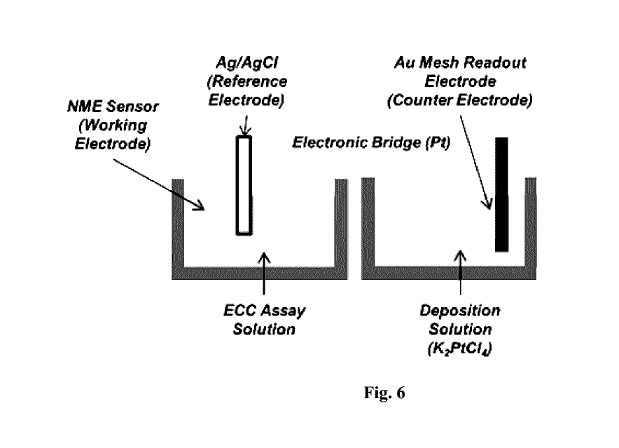

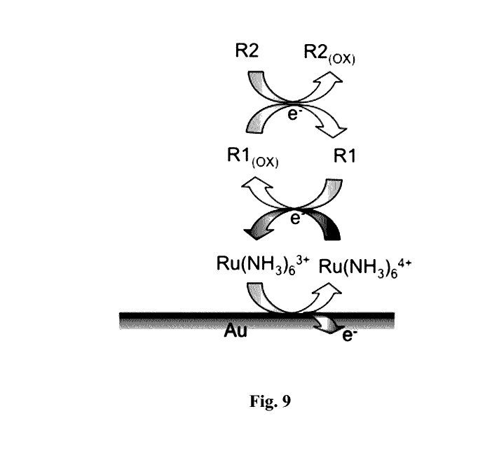

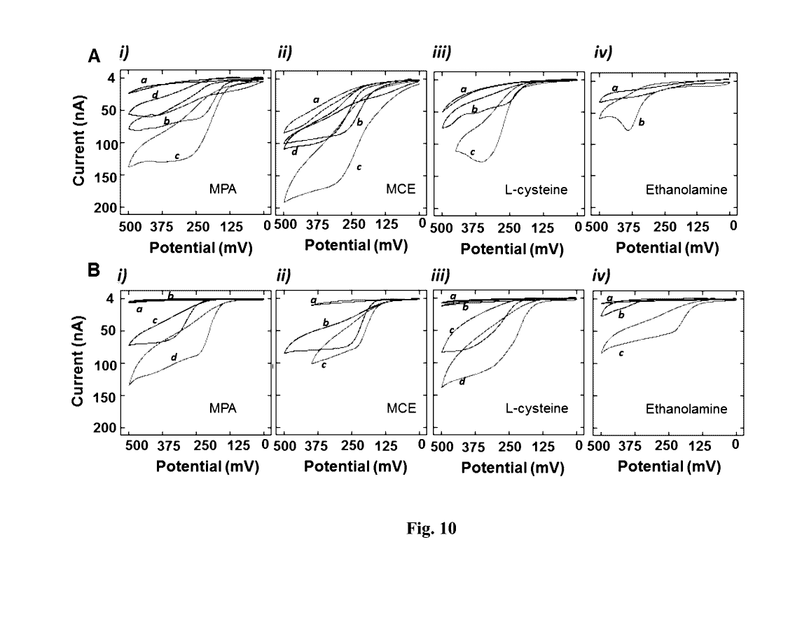

Panel (B) shows comparison of ECC reporter systems using different reducing agents at DNA- or MCH-modified NMEs. (i) a,b: NMEs modified with MCH only; c,d: DNA modified NMEs with MPA+RuHex and MPA+RuHex+cysteamine respectively. (ii) a: NMEs modified with MCH only, with mercaptoethanol (MCE); b,c: DNA modified NMEs, with MCE+RuHex and MCE+RuHex+cysteamine respectively. (iii) a,b: NMEs modified with MCH only, with L-cysteine and L-cysteine+RuHex respectively; c,d: DNA modified NMEs, with L-cysteine+RuHex+TCEP and L-cysteine+RuHex+cysteamine respectively. (iv) a: NMEs modified with MCH only; b,c: DNA modified NMEs with ethanolamine+RuHex and ethanolamine+RuHex+cysteamine respectively. It is noted that any one or more the systems and processes discussed in the referenced figures can be combined. For example, systems described in FIG. 1A or FIG. 1B can be combined with the setup described in FIG. 6, and using one of the ECC reporter systems described in FIG. 9 or 10.

### DETAILED DESCRIPTION

Disclosed herein are methods and systems to detect low-concentration analytes by transducing small electrochemical currents into easily perceived, high-contrast visual changes using a new approach termed electrocatalytic fluid displacement (EFD).

1. Overview of Electrocatalytic Fluid Displacement (EFD)

In one aspect, the EFD approach is based at least in part on the electrodeposition of a metal catalyst, such as platinum, that catalyzes peroxide (e.g., hydrogen peroxide) decomposition. An illustrative example of this electrodeposition process is shown in FIG. 1B, where Pt⁴⁺ ions are reduced to Pt⁰ on a mesh electrode. In some implementations, the substrate may be replaced with other peroxide compounds such as sodium peroxide (Na₂O₂). It is understood that other metal catalysts may also be suitable. For example, many transition metals, metal ions, and compounds may be used as catalysts. These include, but are not limited to gold, silver, palladium, Fe²⁺, Ti³⁺, and MnO₂.

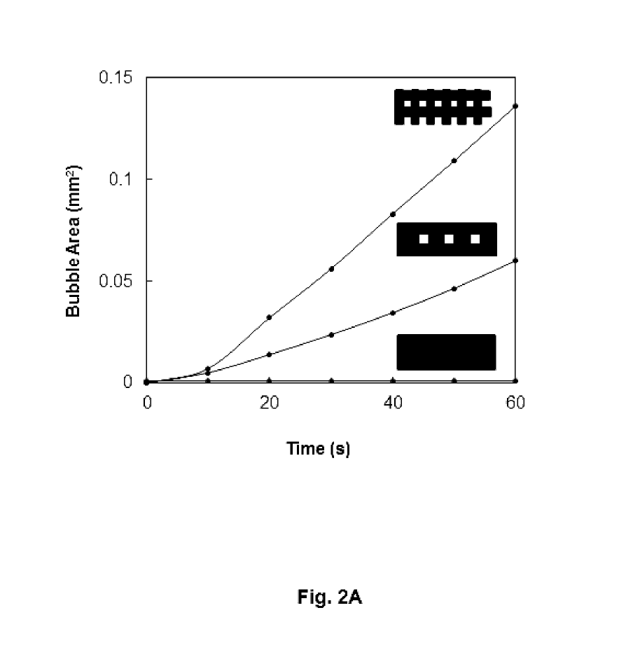

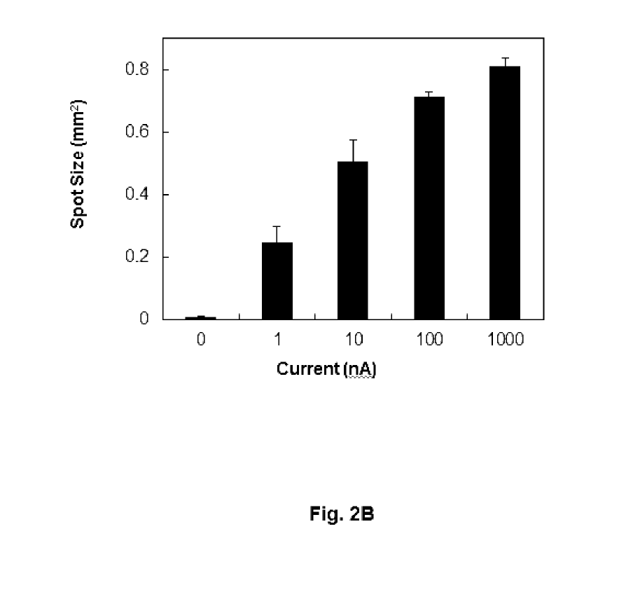

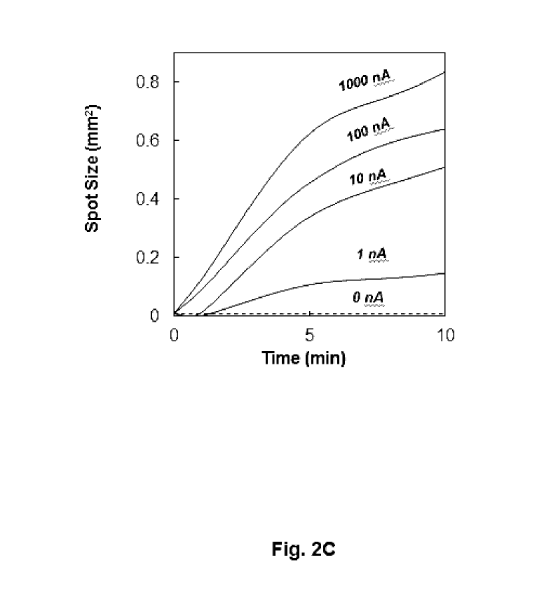

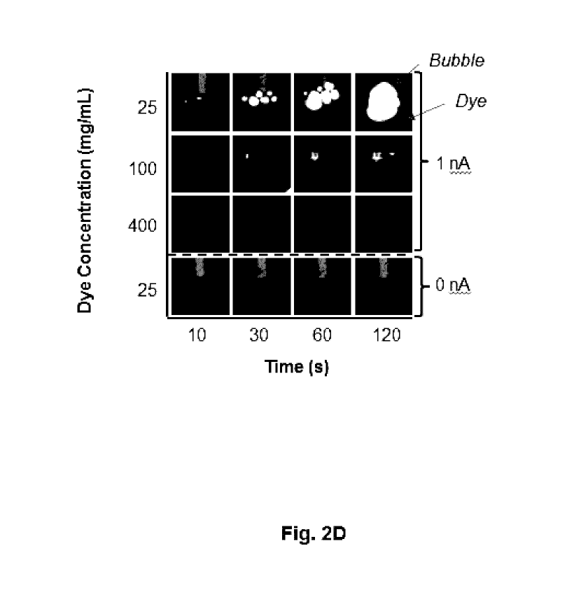

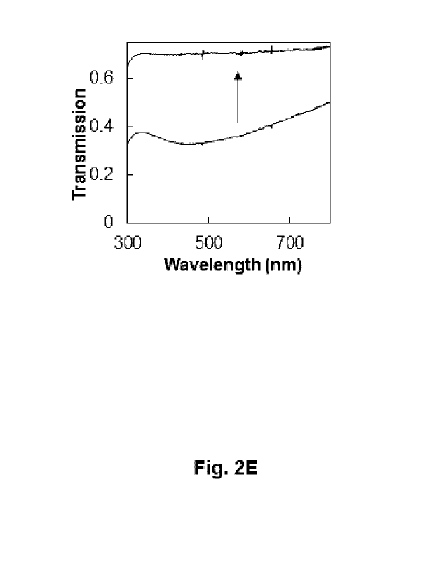

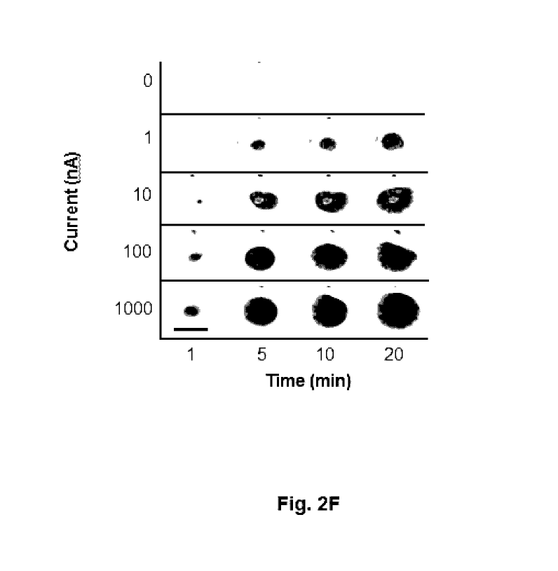

In some implementations (which may be combined and used in conjunction with other implementations discussed herein), a mesh electrode at the bottom of a chamber serves as a template for electrodeposition of a metal catalyst upon the application of a current. Upon the introduction of hydrogen peroxide solution in the chamber, the metal catalyst catalyzes the decomposition of peroxide into water and oxygen, which forms a merging bubble (e.g., FIG. 2A, FIG. 2B). In some implementations, the growing bubble displaces a dye present in the solution to reveal a colored spot in the chamber beneath the electrode. Any dye that provides sufficient optical density to create contrast and whose chemistry does not interfere with the EFD reaction is suitable. Such dyes may be molecular dyes (e.g., light absorbing molecules) or scattering-based pigments (such as the white pigment consisting of titanium particles used in FIG. 2F). The dye/pigment solution can also contain a mixture of absorbing and scattering components.

In some implementations (which may be combined and used in conjunction with other implementations discussed herein), the growing bubble displaces peroxide, which causes an index mismatch at a diffraction grating patterned in the underside of the chamber lid. Incident white light is diffracted into its component colors causing a structural color change. In certain implementations, the underside of the lid of the device may be patterned with other photonic structures such that the growing bubble induces either the appearance or disappearance of other forms of structural color including coherent scattering, incoherent scattering and iridescence.

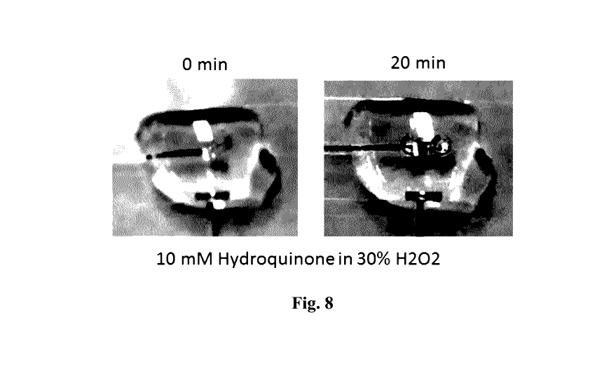

In some implementations (which may be combined and used in conjunction with other implementations discussed herein), the catalyst induces a change in the light absorption properties or color of a dye molecule or pigment particle in solution. For example, many transition metal catalysts will catalyze a color change in the presence of a mixture of hydrogen peroxide and pigments such as hydroquinone, p-aminophenol, or 3,3′,5,5′-tetramethylbenzidine (TMB). See, e.g., FIG. 8 and related discussion provided herein.

In some implementations, to sense a nucleic acid sequence in a sample, the EFD system is connected to a sensor electrode that includes an immobilized nucleic acid probe. In some implementations, a nanostructured microelectrode (NME) is used, which acts as an ultrasensitive electrochemical biosensor (e.g., FIG. 1B). NMEs are electrodes, which are nanotextured and thus have an increased surface area. Preferred NMEs are comprised of a noble metal, such as but not limited to gold, platinum, palladium, silver; alloys of noble metals, such as but not limited to, gold-palladium, silver-platinum; conducting polymers; metal oxides; metal silicides; metal nitrides; or combination of any of the above. NMEs of the above-described materials are highly conductive and form strong bonds with probes, such as nucleic acids. Preferred NMEs have a height in the range of about 0.5 to about 100 microns (um), for example in the range of about 5 to about 20 microns (e.g., 10 microns); a diameter in the range of about 1 to about 10 microns; and have nanoscale morphology (e.g., are nanostructured on a length scale of about 1 to about 300 nanometers and more preferably in the range of about 10 to about 20 nanometers). NMEs can be any of a variety of shapes, including hemispherical, irregular, spiky, cyclical, wire-like, dendritic, or fractal. The surface of an NME may be further coated with a material, which maintains the electrode's high conductivity, but facilitates binding with a probe. For example, nitrogen containing NMEs (e.g., TiN, WN, or TaN) can bind with an amine functional group of the probe. Similarly, silicon/silica chemistry as part of the NME can bind with a silane or siloxane group on the probe.

The NME sensors may be fabricated on silicon substrates using a two-step electrodeposition process as previously described. For example, in a gold nanostructured microelectrode, the gold microstructures protrude from the surface and reach into solution which increases the probability of interaction with the target molecules. The microstructures are decorated with a second layer of finely or roughly nanostructured gold. These nanoscale structures on the microelectrode surface with varying roughness enable additional surface area to immobilize probes and maximize sensitivity by enhancing the hybridization efficiency of the probe and target. Examples of such NME sensors are described in U.S. Pat. No. 8,888,969, which is hereby incorporated herein by reference in its entirety.

In some implementations, a multi-pronged strategy may be used to reduce (e.g., minimize) the current in the absence of target analyte. In an implementation, the sensors are functionalized using a charge-neutral probe, and the current read using a novel electrochemical assay described herein.

In implementations where the target analyte is a nucleic acid sequence, the sensors may be functionalized with thiolated nucleic acid probes (e.g., ribonucleic acids (RNA), deoxyribonucleic acids (DNA), or analog thereof, including, for example a peptide nucleic acid (PNA), locked nucleic acids, or phosphorodiamidate morpholino oligomers. In certain such implementations, the probe is a peptide nucleic acid (PNA) probes complementary to the target sequence. PNA is a synthetic nucleic acid analog which has a neutral charge. This neutral charge reduces or minimizes the background current and increases the signal-to-noise ratio. After target nucleic acid hybridization and washing, the sensor electrodes are subjected to an electrochemical redox reporter system in which an electrical current is generated per each nucleic acid hybridization event. The electrical current from the sensor drives the electrodeposition of platinum on an EFD reporter electrode, which results in degradation of the peroxide on the electrode forming a bubble that displaces the dye to reveal a colored spot beneath the electrode. As discussed above, in an alternative implementation, the growing bubble displaces peroxide, which causes an index mismatch at a diffraction grating patterned in the underside of the chamber lid. Incident white light is diffracted into its component colors causing a structural color change. When the target sequence is not present, the current is too low to deposit a sufficient amount of platinum to catalyze bubble formation or growth and no color change occurs.

As discussed above, the system disclosed herein may be implemented for the detection of other bioanalytes such as proteins and small molecules. In some implementations, the analyte of interest may be a small molecule, including but not limited to a therapeutic drug, a drug of abuse, environmental pollutant, and free nucleotides. In such implementations, the probe may be an aptamer configured to bind the small molecule. In some implementations, the analyte of interest may be a protein or protein fragment. In such implementations, the probe may be an aptamer configured to bind to the protein or protein fragment. In certain implementations, the analyte of interest may be an uncharged molecule. In certain implementations, the analyte is a small molecule with a molecular weight of less than about 500 Daltons.

In some implementations, the electrochemical reporter system is an electrocatalytic reporter pair comprising Ru(NH₃)₆³⁺ and Fe(CN)₆³⁻. Ru(NH₃)₆³⁺ is electrostatically attracted to a target analyte, such as a negatively-charged phosphate backbone of nucleic acid sequence, that binds to the probes immobilized on the surface of sensor electrodes and is reduced to Ru(NH₃)₆² when the electrode is biased at the reduction potential. The Fe(CN)₆³⁻ present in solution chemically oxidizes Ru(NH₃)₆²⁺ back to Ru(NH₃)₆³⁺ allowing for multiple turnovers of Ru(NH₃)₆³⁺, which generates an high electrocatalytic current. This reporter system may be used in conjunction with differential pulse voltammetry.

In some implementations, a DC potential may be used for readout instead voltammetry (although voltammetry may be suitable in some implementations). Since the Ru(NH₃)₆³⁺ and Fe(CN)₆³⁻ system produce high background currents using DC potential amperometry, a novel Electrochemical-Chemical-Chemical (ECC) redox reporter system is provided that eliminates or reduces interfering redox reactions near the potential of interest. Accordingly, in some implementations, the electrochemical reporter system is the novel ECC redox cycle reporter system described below.

2. ECC Redox Cycle Reporter System

Further provided herein is a new ECC redox reporter system and method for using the same. In one aspect, the new ECC redox cycle reporter system radically amplifies the current generated from target nucleic acid hybridization. To the best of the inventors' knowledge, this is the first reported use of ECC for the detection of nucleic acids to date. In one aspect, the ECC system includes a redox molecule that is electrostatically attracted to the backbone of the bound target nucleic acids and reducing agents which regenerate the original form of the redox molecule in order to amplify the signal. See, e.g., FIG. 9 and related description for an illustrative depiction of the ECC redox reporter system according to one implementation. The ECC amplification system enables readout using a DC potential, which is much simpler than standard electrochemical techniques, such as voltammetry (which requires a potential sweep and thus more complicated electronics). Accordingly, in some implementations, in order to simplify the electronics in a disposable device, a DC potential is used for readout. When using a DC potential, it is desirable to eliminate or reduce the contribution from unwanted redox reactions occurring at nearby potentials. Thus, in some implementations, the ECC amplification chemistry is a redox reporter system in which there are no or minimal interfering redox reactions near the potential of interest, enabling DC readout with low background currents.

In some implementations, the reducing agents are not oxidizable at the electrode surface in order to reduce the background current. In some implementations, the reducing agents are not oxidizable or reducible by Ru(NH₃)₆³⁺ or Ru(NH₃)₆⁴⁺. In some implementations, the relationship between the formal potentials of the ECC system species may be characterized as follows:

Electrode<Ru(NH₃)₆³⁺,Ru(NH₃)₆⁴⁺,<R₁,R_{1(OX)}, or R₂, R_{2(OX)}.

Reducing agents which may be used in the ECC system include, but are not limited to, 3-mercaptopropionoic (MPA) acid, cysteamine (Cys), mercaptoethanol (MCE), cysteine, tris(2-carboxyethyl)phosphine (TCEP), and ethanolamine. In some implementations, signal amplification using ECC is achieved using a single reducing agent as opposed to a pair of reducing agents, although a larger concentration of reducing agent must be used. However, systems with two reducing agents have been found to produce lower background currents. Accordingly, in some implementations, signal amplification using ECC is achieved using a pair of reducing agents. Pairs of reducing agents which may be used in the ECC system include but are not limited to: 3-mercaptopropionoic (MPA) acid and cysteamine (Cys); mercaptoethanol and cysteamine; cysteine and tris(2-carboxyethyl)phosphine (TCEP); ethanolamine and TCEP; cysteine and cysteamine; and ethanolamine and cysteamine. See FIG. 10 comparing different reducing agents suitable for use in the ECC system.

In some implementations, the ECC redox system employs Ru(NH₃)₆³⁺, mercaptopropionic acid (MPA), and cysteamine. Ru(NH₃)₆³⁺ is electrostatically attracted to the negatively-charged phosphate backbone of the bound target nucleic acids. Upon the application of an appropriate potential (250 mV in this example), Ru(NH₃)₆³⁺ is oxidized to Ru(NH₃)₆⁴⁺. The MPA present in solution chemically reduces Ru(NH₃)₆⁴⁺ back to Ru(NH₃)₆³⁺, allowing for multiple turnovers of Ru(NH₃)₆³⁺, which generates a high electrocatalytic current. This signal is further amplified by cysteamine, another reducing agent, which is chemically oxidized to cystamine by reducing the oxidized-form of MPA (R-S-S-R) back to its reduced form (R-SH).

In some implementations, in the presence of a target analyte (e.g., nucleic acids), the current drives the electrodeposition of platinum on the EFD electrode which catalytically forms a bubble that displaces the dye to reveal the colored spot. When the target analyte is not present, the current is too low to deposit a sufficient amount of platinum to catalyze bubble growth and no color change occurs (FIG. 2B).

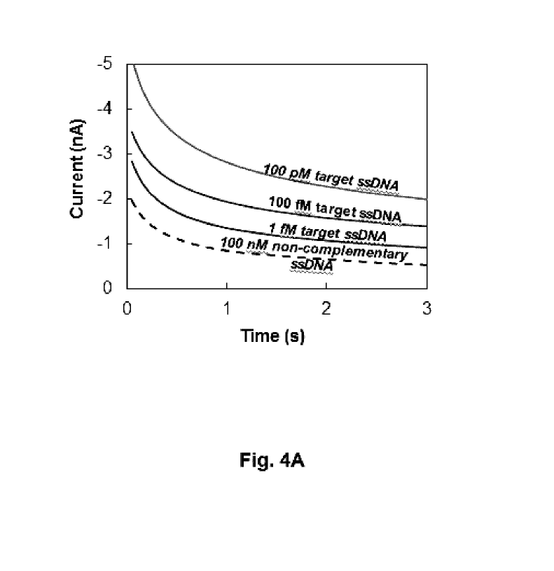

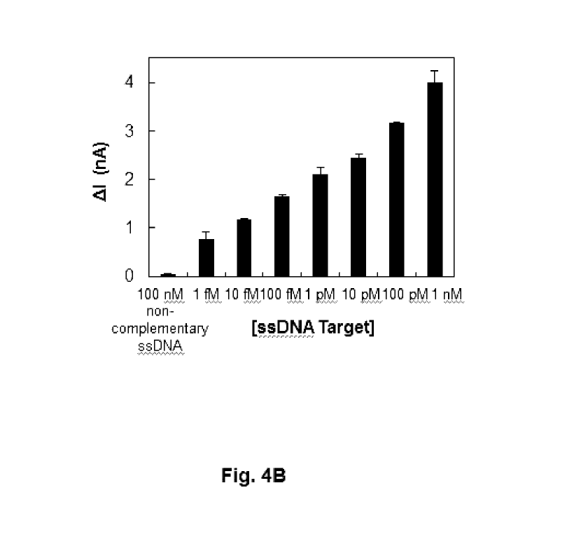

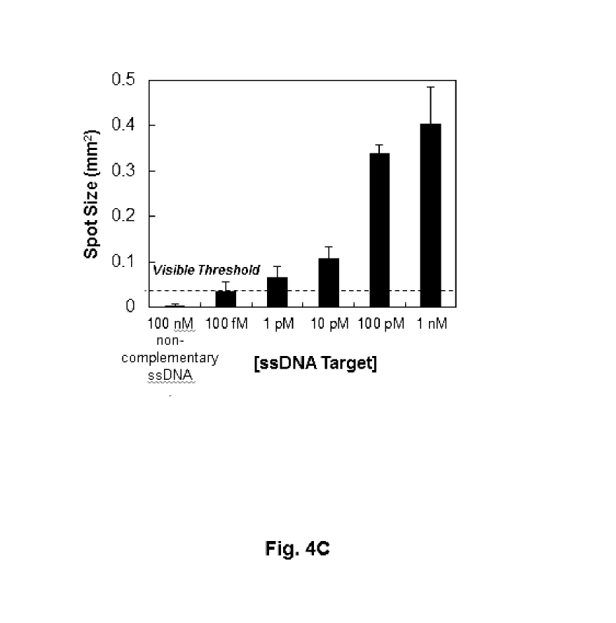

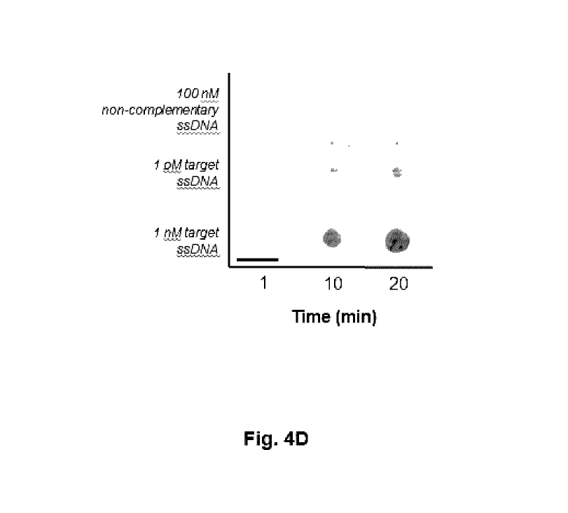

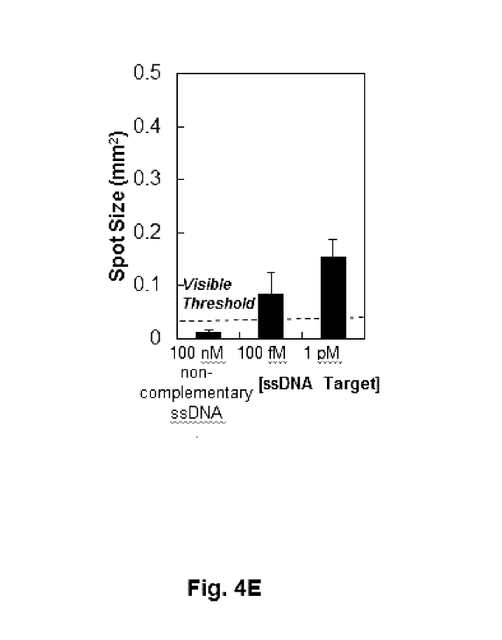

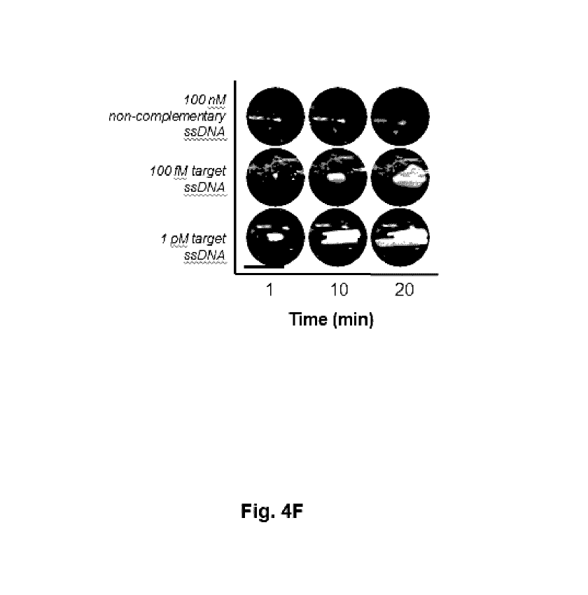

In some implementations (which may be combined with any of the above-referenced implementations), in the presence of a target analyte (e.g., nucleic acids), the current drives the electrodeposition of platinum on the EFD electrode which catalytically forms a bubble that displaces peroxide, which causes an index mismatch at a diffraction grating patterned in the underside of the chamber lid. Incident light is diffracted into its component colors causing a structural color change. When the target sequence is not present, the current is too low to deposit a sufficient amount of platinum to catalyze bubble growth and no detectable color change occurs (FIGS. 4A, 4D, and 4E).

3. A Comparative Model of Color Change Resultant from EFD

Catalytic electrochromic transduction methods offer significant signal amplification needed for transducing the ultra-low currents generated by the ECC assay compared to direct electrochromic reduction. To study the prospective performance of this approach, we calculated the predicted time required to induce a visible color change using a variety of transduction strategies.

We illustrate the challenge of directly inducing a color change by considering the example of electrodepositing an optically-discernible quantity of metal. A current of about 1 nA applied for about 10 s supplies about 6×10¹⁰ electrons which can turnover a maximum of 6×10¹⁰ molecules. Even under the generous assumption that a single molecular layer is visible, given an atomic radius of about 1 Å, this yields a spot of only about 50 μm×about 50 μm. This is too small to be easily visible to the naked eye as the spatial resolution of human eyesight is approximately about 100 to about 200 μm.

The EFD detection systems and methods provided herein are capable of amplifying, by orders of magnitude, the color change per charge. In one aspect, a catalyst, such as platinum, is electrodeposited to turn on the colorimetric reaction. By depositing a catalyst, each electron effectively converts multiple molecules, amplifying the color transformation. However, as FIG. 1C shows, even the catalytic reduction of an electrochromic compound in bulk solution requires exceedingly long times to induce a visible change. Assuming a 50 μm tall chamber with a 200 μm diameter window filled with enough pigment, with the absorbance of malachite green, to give an OD of about 1, it would take about over 4 hours to turnover the compound using the platinum deposited from a current of about 1 nA.

Thus, in some implementations, instead of catalytic reduction of a solution-based pigment, a gaseous substance is used, as an equivalent molar amount of gas occupies a much larger volume than a liquid. At standard temperature and pressure (STP), the volume of about 1 mole of gas is about 22.4 L, which is about 3 orders of magnitude larger than a mole of liquid H₂O (18 mL). Platinum is an excellent catalyst for the decomposition of hydrogen peroxide to form oxygen and water. As FIG. 1C shows, the catalytic production of a visible bubble that fills the same window requires under about 3 minutes, about over 80 times faster than catalytic reduction of an electrochromic dye in solution. The electrocatalytic bubble formation may be converted into a colorimetric change by actuating a fluid to modulate the optical density (OD) of the readout window.

4. Optimization of Device Geometry

In one aspect, the electrocatalytic fluidic displacement approach may be implemented using a rectangular gold electrode patterned on a glass substrate which sits at the bottom of a chamber. The chamber may be any suitable size, e.g., a circular chamber of about 50 μm tall by about 1.5 mm wide. After depositing platinum for about 10 s at about 1 nA, a hydrogen peroxide solution is introduced and the rate of bubble growth is measured using, e.g., a microscope (See, e.g., FIG. 2A). Bubbles are formed preferentially at the electrode edges, without observable rapid growth.

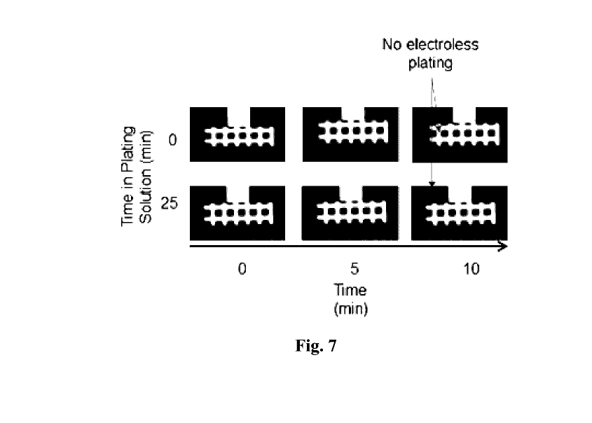

In one implementation, mesh shaped electrodes with increased ratios of edges to surface area were designed and fabricated to test the enhancement provided by edges. About 1 nA current was applied for about 10 s to deposit platinum and the rate of bubble growth was recorded (FIG. 2A). The rate of bubble evolution increased with increasing numbers of edges. The highest density mesh, with about 3.4 times the edge to surface area ratio of the rectangular electrode, provided the fastest bubble growth. No bubbles formed when no current was applied as no platinum was electrodeposited. Bubble growth was not observed after immersing the device in platinum solution for about 25 minutes, indicating that platinum is not deposited via electroless deposition (see, e.g., FIG. 7).

The average growth of the bubble for various applied currents was measured using the high density mesh electrodes. FIG. 2B shows the average bubble area measured after about 20 minutes as a function of electrodeposition current according to one implementation. FIG. 2C shows the bubble growth over time according to one implementation. After about 20 minutes, application of a current of about 1 nA for about 10 s yielded a bubble with an area of about 0.25 mm², which was visibly detectable.

5. Electrocatalytic Fluidic Dye Displacement

In some implementations, to induce a visible color change that is easily interpretable by the end-user, a bubble is used to displace an opaque dye that obscures a colored spot beneath the readout window. As the chamber fills with oxygen, the colored spot is revealed.

In this implementation, increasing the dye concentration increased the opacity of the dye, but also increased its viscosity. At higher viscosities, bubble formation was inhibited (See, e.g., FIG. 2D). Upon optimization of the dye concentration, it was found that using a concentration of about 25 μg/mL of the dye allowed for sufficient optical density to conceal the colored spot while promoting bubble growth (FIG. 2D).

In some implementations, to determine the minimum visibly detectable current, platinum was deposited at various rates for about 10 s and the exposed area of the blue spot was measured (See, e.g., FIG. 2F). Using a deposition current of about 1 nA, the spot area grew to about 0.09 mm² in about 5 minutes (FIG. 2F). The exposed area expanded to about 0.24 mm² in about 20 minutes. No bubble growth was observed when platinum was not electrodeposited (FIG. 2F).

The spatial resolution of human eyesight is about 200 μm, making the smallest visible area approximately about 200 μm×about 200 μm or about 0.04 mm². Thus, the spot area of about 0.09 mm² obtained from a current of about 1 nA after about 5 minutes is visible to the naked eye.

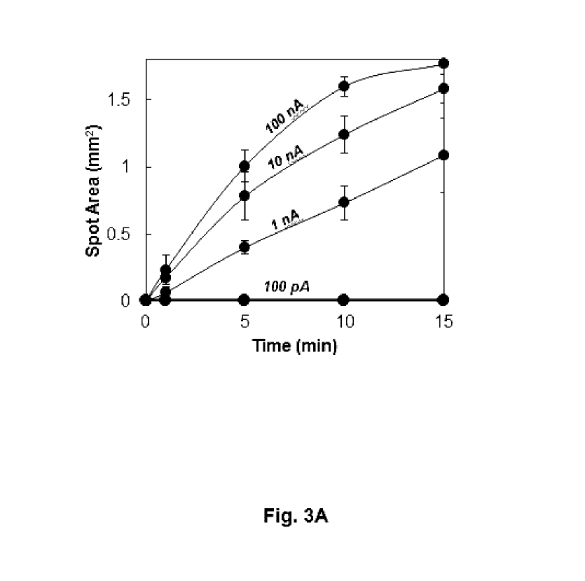

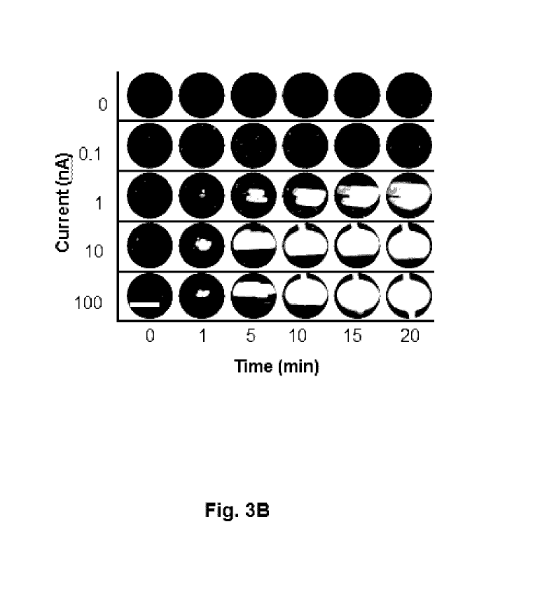

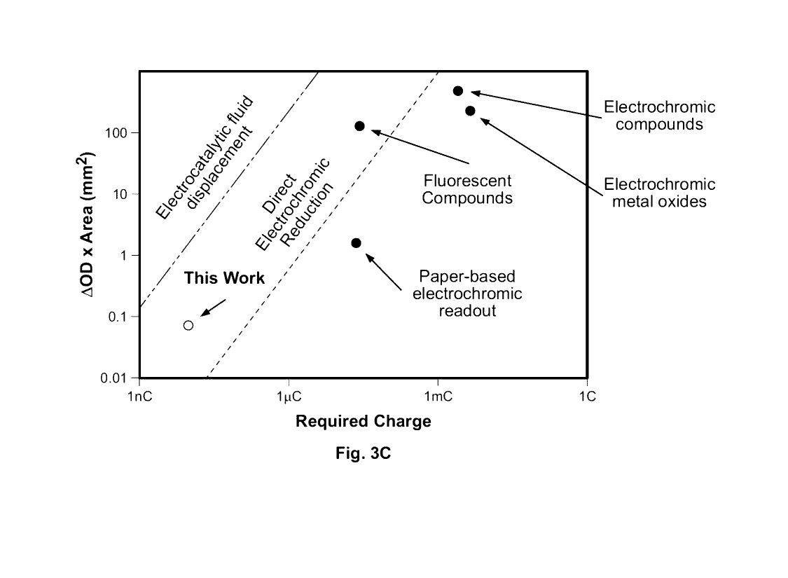

To quantify the performance of the device, the coloration efficiency (CE) may be calculated as follows:

$$CE = \frac{Δ OD \cdot A}{Q} , \qquad ( 1 )$$

in which OD is optical density, Q is the charge required for switching [C] (Coulomb), and A is the spot area [cm²]. The coloration efficiency is a metric to quantify the efficiency of converting an electrical current into a colorimetric change. In this implementation, the optical density was measured before and after switching and turned out to be about 0.27 (FIG. 2E). Given a switchable area of about 0.24 mm² after about 20 minutes using a current of about 1 nA applied for about 10 s, this device has a coloration efficiency of about 6.48×10⁴ cm² C⁻¹. FIG. 3C compares the switchable area as a function of charge for devices with the highest reported coloration efficiencies for a range of readout strategies according to some implementations. Given the previous records of about 2.6×10⁴ cm² C⁻¹ for fluorescent polymers and about 9.3×10² cm² C⁻¹ for non-fluorescent electrochromic compounds, a coloration efficiency of about 6.48×10⁴ cm² C⁻¹ is, to our knowledge, the highest reported value in the literature for an electrochromic device.

6. Electrocatalytic Fluidic Induction of a Structural Color Change

As optical absorbance increases with path length, the readout window may need to be sufficiently tall for the dye to obscure the colored spot beneath. As such, the response time of a colorimetric device based on dye displacement can vary depending on the path length as the bubble needs to grow large enough to reach the chamber ceiling.

According to one aspect, by patterning substrates with feature sizes on the order of the wavelength of light, it is possible to produce vibrant structural colors. Examples of this include diffraction gratings and iridescence. The color of the substrate can be modified by matching the index refraction between a second medium and the substrate. By exploiting a structural color change, the readout turnaround time can be decreased. As structural color changes rely on the index matching at an interface, the color change is largely independent of the path length through the index-matching medium. Thus, a vibrant color change is expected, using a device with a much smaller channel height than required when using dye displacement. As the substrate provides the color, there is no need to increase the opacity of the peroxide by introducing additional compounds which might interfere with the reaction. In one implementation of this approach, a diffraction grating is patterned into the underside of the PDMS lid affixed to the top of the device with a channel about 7 μm tall. As the index of refraction of peroxide (n=1.35) is similar to that of PDMS (n=1.4), the diffraction grating is invisible to incoming light when the device is initially loaded with peroxide. As the bubble is formed, the peroxide is replaced with O₂ which has an index of refraction of 1. This index mismatch between the bubble and PDMS unveils the diffraction grating. The incident white light is diffracted into its component colors to reveal the circular spot. In certain implementations, the change or appearance of diffraction induced by this refractive index change may be read out by the spatial pattern of light spots reflected or transmitted from a monochromatic source such as a laser in a barcode scanner.

FIG. 3A shows the growth of the colored spot using the diffraction grating approach and FIG. 3B shows the corresponding images of the spot over time according to some implementations. As the bubble grew, white light began to diffract into its component colors. The window turns from optically transparent (which appeared as black due to a black background) to cyan as light at that wavelength was diffracted towards the camera. Using a deposition current of about 1 nA, the spot size was about 0.06 mm² after about 1 minute, which was visible by eye. This spot grew to about 0.36 mm² and about 1.1 mm² by about 5 and about 15 minutes respectively. Given a spot size of only about 0.1 mm² after about 5 minutes using electrocatalytic fluidic displacement of a dye, the structural color spot of about 0.36 mm² was over about 3 times larger in the same time frame. No spot formed when no current was applied FIG. 3B.

7. Colorimetric Readout of ssDNA

In one implementation, to test the capability of the EFD device to detect biomarkers, NME sensors are connected in serial to the EFD readout chip and with ssDNA. As an initial characterization of the ECC assay, the sensors were challenged with serial dilutions of ssDNA. The corresponding currents were measured after applying about 250 mV (FIG. 4A). The average peak current decreases with target ssDNA concentration giving a detection limit of about 1 fM (FIG. 4B). The current generated from about 100 nM non-complementary ssDNA was less than about 2 nA, which is similar to the background current, indicating this readout method is specific.

In some implementations, in order to demonstrate colorimetric readout of biomarkers, the assay was coupled to a readout device and the sensors were challenged with serial dilutions of ssDNA. The NME sensors were immersed in the ECC solution and the EFD readout device in the platinum electrodeposition solution, thereby connecting the sensors to the EFD device. However, other methods of coupling the sensors to the EFD device may be used. A platinum wire electrode immersed in the ECC solution was connected to a second platinum electrode in the electrodeposition bath, to bridge electronically the sensor and readout device. See, e.g., FIG. 6 for an illustrative setup. Other suitable means for bridging the sensor and the EFD readout device may be used without departing from the scope of the disclosure. The EFD readout device acted as the counter electrode for the entire system (FIG. 1B). After application of a current about 250 mV for about 10 s to the NME, peroxide was introduced into the EFD chip and measured the rate of color formation (FIG. 4C). A detection limit of about 1 pM was found after about 10 minutes with an average spot size of about 0.068 mm² (FIG. 4C). To our knowledge, a detection limit of about 1 pM is the lowest reported limit of detection for colorimetric detection of ssDNA using an electrochemical sensor. No visible spot was observed when the sensors were challenged with about 100 nM of non-complementary ssDNA indicating a specificity discrimination ratio of about 1×10⁵ (FIG. 4D).

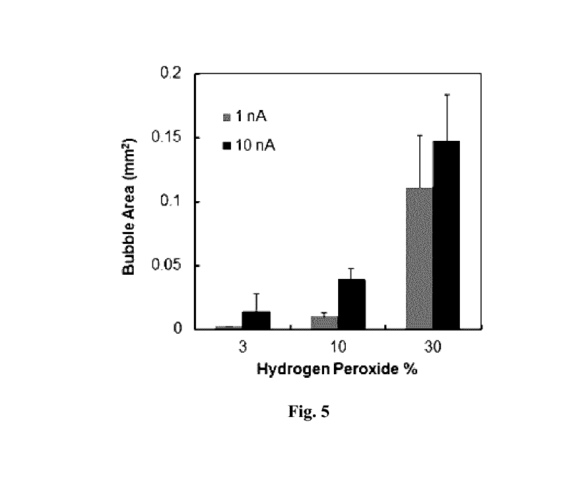

In one implementation, the peroxide concentration was optimized to minimize bubble formation from currents at the background level. Bubble growth at low currents could be suppressed using about 10% peroxide (FIG. 5). After challenging the devices with ssDNA, the growth of the diffracting area was measured (FIG. 4E). FIG. 4f shows the corresponding images of the growth of the visible spot over time. Using 1 pM complementary ssDNA, the spot size was about 0.15 mm² after about 10 minutes. In that same time frame, the spot using dye displacement was about 0.068 mm² which is about 2 times smaller. Using this method, about 100 fM of ssDNA was also detectable by eye with an average spot size of about 0.085 mm² (FIG. 4E). No spot was visible with about 100 nM non-complementary ssDNA (FIG. 4F).

In one implementation, the rate of color change using direct colorimetric readout was calculated under the assumption that a channel about 50 μm tall by about 200 μm wide was filled with enough electrochromic dye to give an OD of about 1. It was further assumed that a high molar absorptivity of about 1×10⁷ M⁻¹m⁻¹ which is similar to that of malachite green. The time needed to turn over the dye in the channel was calculated using the catalysis rate of platinum.

In one implementation, the rate of bubble formation was calculated using electrocatalytic fluidics in a chamber that is about 50 μm tall with a about 200 μm width. The rate of oxygen formation was calculated using the catalysis rate of platinum. The onset of bubble formation occurred as peroxide in the chamber was saturated with oxygen. It was assumed that the bubble is visible once it grows to the volume of the chamber.

| Description | Value |
|---|---|
| Channel Height | about 50 μm |
| Channel Width | about 200 μm |
| Solubility of Oxygen in Water | about 7.6 mg/L |
| Catalysis rate of platinum | about 8.84 × 10⁻⁴ mol |
| | s⁻¹ m⁻² |
| Hydrogen Peroxide Concentration | about 15% |
| Molar absoprtivity of the | about 1 × 10⁷ M⁻¹ m⁻¹ |
| electrochromic dye | |
| Optical Density (OD) of the Dye in | about 1 |
| Channel | |

In one implementation, the device was fabricated using standard photolithographic methods. Briefly, electrodes were patterned on a glass substrate. The device was passivated using SU-8 2002 (Microchem, Newton, Mass.) and apertures were patterned to expose the electrodes below. The channel was fabricated by patterning SU-8 3050.

In one implementation, the electrode was immersed in K₂PtCl₄ and connected to an Epsilon potentiostat (BASi West Lafayette, Ind.) using a 3-electrode setup with a Ag/AgCl reference electrode and a Pt counter electrode. Using chronopotentiometry, various currents were applied for about 10 s. After electrodeposition, the device was washed thoroughly with H₂O and covered with a PDMS lid.

In one implementation, about 100 μL of white dye (Liquitex Titanium White Ink) was centrifuged for about 5 minutes at about 15 000 g. The supernatant was removed and replaced with about 4004 of about 30% H₂O₂ (Sigma). The dye (about 25 pg/mL) was introduced into the channel and the amount of bubble generation was measured over time using a camera (Canon).

In one implementation, a diffraction grating was patterned in PDMS by curing PDMS on a DVD-R. The PDMS diffraction grating lid was removed and attached to the device with an about 7 μm tall channel. About 27% H₂O₂ with about 1% pluoronic (Sigma) was introduced into the device and color changes were measured over time using a camera (Canon).

In one implementation, six inch silicon wafers were passivated using a thick layer of thermally grown silicon dioxide and coated with a Ti adhesion layer of about 25 nm. A gold layer of about 350 nm was deposited on the chip using electron-beam-assisted gold evaporation which was again coated with about 5 nm of Ti. The electrodes were patterned in the metal layers using standard photolithography and a lift-off process. A layer of about 500 nm of insulating Si₃N₄ was deposited using chemical vapor deposition. Apertures of about 5 μm were etched at the tips of the metal leads using standard photolithography. Contact pads (about 0.4 mm×about 2 mm contact) were patterned using wet etching as well.

In some implementations, chips were cleaned by sonication in acetone for about 5 min, rinsed with isopropyl alcohol and DI water, and dried with nitrogen. Electrodeposition was performed at room temperature. Apertures of about 5 μm on the fabricated electrodes were used as the working electrodes and were contacted using the exposed bond pads. Nanostructured microelectrodes sensors were electrodeposited in a solution of about 50 mM HAuCl₄ and about 0.5 M HCl using DC potential amperometry at about 0 mV for about 100 s. After washing with DI water and drying, the sensors were coated again with a thin layer of Au to form nanostructures by plating at about −450 mV for about 10 s.

In one implementation, an aqueous solution containing about 1 μM of probe (5′-GGT CAG ATC GTT GGT GGA GT-3′) was mixed with about 10 μM of aqueous Tris(2-carboxyethyl)phosphine hydrochloride solution and then the mixture was left for overnight to cleave disulphide bonds. After mixing about 100 nM of 6-mercaptohexanol (MCH) to this probe solution mixture, about 20 μL was pipetted onto the chips and incubated for about 3 h in a dark humidity chamber at room temperature for probe immobilization. The chips were then washed thrice for about 5 min with 0.1×PBS at room temperature. The chips were then treated with about 1 mM MCH for an hour at room temperature for back filling. After washing, the chips were challenged with different concentration of targets for about 30 min at room temperature. After hybridization, the chips were washed thrice for about 5 min with about 0.1×PBS at room temperature and the electrochemical scans were acquired.

In some implementations, electrochemical experiments were carried out using a Bioanalytical Systems Epsilon potentiostat with a three-electrode system featuring a Ag/AgCl reference electrode and a platinum wire auxiliary electrode. Electrochemical signals were measured in a Tris buffer solution (about 50 mM, pH of about 9) containing about 10 μM [Ru(NH₃)₆]Cl₃, about 0.5 mM 3-mercaptopropionoic acid (MPA), and about 0.5 mM cysteamine (Cys). DC potential amperometry (DCPA) signals were obtained at about +250 mV for about 10 s. Signal changes, ΔI, were calculated with ΔI=I_{c}−I₀ (where I_{c} is the current at a given concentration and I₀ is the current without analyte).

Variations and modifications will occur to those of skill in the art after reviewing this disclosure. The disclosed features may be implemented, in any combination and subcombination (including multiple dependent combinations and subcombinations), with one or more other features described herein. The various features described or illustrated above, including any components thereof, may be combined or integrated in other systems. Moreover, certain features may be omitted or not implemented. All references cited are hereby incorporated by reference herein in their entireties and made part of this application.

Examples of changes, substitutions, and alterations are ascertainable by one skilled in the art and could be made without departing from the scope of the information disclosed herein. All references cited herein are incorporated by reference in their entirety and made part of this application.

What is claimed is:

1. A detection system for detecting a target analyte in a sample, the system comprising:
a first chamber comprising a sensor electrode capable of presenting a biomolecular probe at the surface thereof, said probe capable of binding the target analyte;
a second chamber comprising a readout electrode electrically coupled to the sensor electrode;
a peroxide solution; and
a metal catalyst.

2. The detection system of claim 1, wherein the first chamber comprises a redox reporter comprising Ru(NH₃)₆³⁺ and a reducing agent, wherein the reducing agent is not oxidizable or reducible by Ru(NH₃)₆³⁺ or Ru(NH₃)₆⁴⁺.

3. The detection system of claim 2, wherein the reducing agent is selected from: 3-mercaptopropionoic (MPA) acid, cysteamine (Cys), mercaptoethanol (MCE), cysteine, tris(2-carboxyethyl)phosphine (TCEP), and ethanolamine.

4. The detection system of claim 2, wherein the reducing agent comprises a combination of agents selected from: 3-mercaptopropionoic (MPA) acid+cysteamine (Cys); mercaptoethanol (MCE)+cysteamine; cysteine+tris(2-carboxyethyl)phosphine (TCEP); ethanolamine+TCEP; cysteine+cysteamine; and ethanolamine+cysteamine.

5. The detection system of claim 1, wherein binding of the target analyte to the probe on the sensor electrode generates an electrical current that results in electrodeposition of the metal catalyst on the readout electrode.

6. The detection system of claim 5, wherein electrodeposition of the metal catalyst on the readout electrode causes decomposition of the peroxide present in the second chamber.

7. The detection system of claim 6, wherein decomposition of peroxide generates oxygen bubbles.

8. The detection system of claim 7, wherein generation of bubbles displaces a dye present in the peroxide solution.

9. The detection system of claim 8, wherein the second chamber comprises a colored spot beneath the readout electrode.

10. The detection system of claim 9, wherein displacement of a dye in the peroxide solution reveals the colored spot beneath the readout electrode.

11. The detection system of claim 1, wherein the sensor electrode is a nanostructured microelectrode.

12. The detection system of claim 1, wherein the readout electrode is a mesh or high-edge-density electrode.

13. The detection system of claim 1, wherein the sensor electrode is electrically coupled to the readout electrode through a platinum wire electrode.

14. The detection system of claim 1, wherein the peroxide solution and metal catalyst are added to the second chamber sequentially.

15. The detection of system of claim 1, wherein the peroxide solution and metal catalyst are added to the second chamber simultaneously.

16. The detection system of claim 1, wherein the metal catalyst is platinum.

17. The detection system of claim 1, wherein the analyte is nucleic acid.

18. The detection system of claim 1, wherein the probe is a peptide nucleic acid probe or a nucleic acid probe.

19. The detection system of claim 1, wherein the second chamber comprises a lid comprising a diffraction grating, wherein generation of bubbles causes an index mismatch at the diffraction grating, causing a structural color change.

20. The detection system of claim 1, wherein the second chamber comprises a lid comprising a photonic structure, wherein generation of bubbles induces the appearance or disappearance of incoherent scattering, coherent scattering or iridescence, causing a structural color change.

21. The detection system of claim 20, wherein the lid is made of material having an index of refraction substantially the same as peroxide.
